(19) United States
(12) Patent Application Publication
(10) Pub. No.: US 2025/0000566 A1
(43) Pub. Date: Jan. 2, 2025

(54) HIGH-FREQUENCY GENERATOR, SYSTEM, METHOD FOR GENERATING A HIGH-FREQUENCY SQUARE-WAVE VOLTAGE, AND USE OF A HIGH-FREQUENCY GENERATOR

(71) Applicant: KARL STORZ SE & Co. KG, Tuttlingen (DE)

(72) Inventor: Thomas HINDING, Tuttlingen (DE)

(21) Appl. No.: 18/748,672

(22) Filed: Jun. 20, 2024

(30) Foreign Application Priority Data
Jun. 30, 2023 (DE) 10 2023 117 342.1

(51) Int. Cl. A61B 18/12; A61B 18/00; H02M 7/219

(52) U.S. Cl. CPC A61B 18/1206; H02M 7/219; A61B 2018/00589; A61B 2018/00601; A61B 2018/00607; A61B 2018/00827; A61B 2018/00892; A61B 2018/0094

(57) ABSTRACT

The invention relates to a high-frequency generator (10) comprising a primary circuit (11) to which an AC voltage can be applied, a secondary circuit (12), a transformer (13) for coupling the primary circuit to the secondary circuit in a manner galvanically isolated from the secondary circuit, and a voltage conversion device (14) which is designed to generate a high-frequency square-wave voltage from the AC voltage transmitted from the primary circuit to the secondary circuit by means of the transformer.

### CROSS-REFERENCE TO RELATED APPLICATIONS

This application claims the benefit under 35 U.S.C. 119(a) to German Patent Application No. 10 2023 117 342.1, filed 30 Jun. 2023, the disclosure of which is incorporated herein by reference in its entirety.

### SUMMARY

The invention relates to a high-frequency generator, comprising a primary circuit to which an AC voltage can be applied, a secondary circuit and a transformer for coupling the primary circuit to the secondary circuit in a manner galvanically isolated from the secondary circuit. The invention further relates to a system comprising a high-frequency generator and a high-frequency apparatus, in particular an electrosurgical instrument for coagulating and/or cutting biological tissue. Furthermore, the invention relates to a method for generating a high-frequency voltage by means of a high-frequency generator. Lastly, the invention relates to the use of a high-frequency generator for an electrosurgical instrument for coagulating and/or cutting biological tissue.

In high-frequency surgery (HF surgery) or electrosurgery, a high-frequency alternating current is passed through a patient's human body in order to specifically damage or cut biological tissue in the body by causing heat.

In minimally invasive endoscopy, electrosurgical coagulation or cutting instruments are used. This requires fast and reliable tissue sealing. Even a small amount of blood is enough to obstruct a surgeon's view and make it difficult or even impossible to operate on the patient. In principle, tissue sealing involves heating tissue using a high-frequency current. As a result of being heated, the tissue is sealed. This makes it possible to immediately stop any bleeding in the tissue that may occur.

The current used for heating must have a certain frequency to prevent nerve irritation. In other words, the nerves are not intended to be stimulated. Typically, appropriate frequencies are in the kHz range.

In order to achieve the required voltage and to achieve galvanic isolation required for safety reasons, a transformer can be used. The transformer is typically operated by means of a half or full bridge on a primary side of the high-frequency generator with a sinusoidal voltage with a frequency that is often at least 150 kHz. A voltage that is galvanically isolated from the primary side can then be applied to a secondary side of the high-frequency generator and can be used, for example, to coagulate or cut tissue. The tissue to be treated must not be heated too strongly or for too long, as this may lead to carbonization or the formation of eschars, which can trigger further uncontrollable bleeding. It is therefore necessary for the high-frequency generator to record a large number of controlled variables, in particular a voltage or a current, in order to generate a signal. In bipolar systems, it is also necessary that radio frequency identification is also available. However, accurate or reliable measurement or control of the voltage is difficult due to the sinusoidal voltage regularly used in the prior art.

The frequency described above is generated by means of a half or full bridge. However, due to their high performance, the components used can only be operated digitally, so they can only be switched on or off. A transformer can ideally be operated with a sinusoidal voltage. “Hard” switching of the transistors used, mostly MOSFETs, can cause many harmonics, i.e., disturbances that can become critical in an EMC test on the one hand and can also make exact measurements of current-voltage phase shifts considerably more difficult and inaccurate on the other hand. At lower frequencies, a delta modulation method can be used. However, this is not possible at higher frequencies because the transistors cannot switch quickly enough.

For example, a high-frequency generator operating with a sinusoidal signal is disclosed in the applicant's German patent application with the official file reference 10 2022 127 935.9, which had not yet been published by the priority date of the present patent application.

The object of the present invention is therefore to provide a high-frequency generator, a system comprising a high-frequency generator and a high-frequency apparatus, in particular an electrosurgical instrument for coagulating and/or cutting biological tissue, a method for generating a high-frequency voltage by means of a high-frequency generator and the use of a high-frequency generator for an electrosurgical instrument for coagulating and/or cutting biological tissue, which overcomes the disadvantages in the prior art and in particular enables improved execution of coagulation or tissue cutting.

This object is achieved by a high-frequency generator having the features of claim 1, by a system comprising a high-frequency generator and a high-frequency apparatus, in particular an electrosurgical instrument for coagulating and/or cutting biological tissue, having the features of claim 16, by a method for generating a high-frequency voltage by means of a high-frequency generator having the features of claim 17, and by the use of a high-frequency generator for an electrosurgical instrument for coagulating and/or cutting biological tissue having the features of claim 21.

The high-frequency generator according to the invention comprises a primary circuit to which an AC voltage can be applied, a secondary circuit, a transformer for coupling the primary circuit to the secondary circuit in a manner galvanically isolated from the secondary circuit, and a voltage conversion device which is designed to generate a high-frequency square-wave voltage from the AC voltage transmitted from the primary circuit to the secondary circuit by means of the transformer.

Advantageously, the high-frequency generator can be used in connection with a high-frequency apparatus, which can in particular be an electrosurgical instrument for coagulating and/or cutting biological tissue. In principle, however, the high-frequency generator can also be used in connection with any high-frequency applications, which can in particular also be located outside the technical field of HF surgery. The high-frequency generator and also the method shall be described below, particularly in connection with a high-frequency apparatus or electrosurgical instrument for coagulating and/or cutting biological tissue, which is in no way intended to be limiting.

According to the invention, the high-frequency generator comprises a primary circuit to which an AC voltage can be applied, a secondary circuit to which a high-frequency apparatus or an electrosurgical instrument for coagulating and/or cutting biological tissue or at least one pair of electrodes of an electrosurgical instrument for coagulating and/or cutting biological tissue can be connected, and a transformer for coupling the primary circuit to the secondary circuit in a manner galvanically isolated from the secondary circuit. Galvanic isolation (also called galvanic decoupling or electrical isolation) is understood to mean avoiding electrical conduction between two circuits between which power or signals are to be exchanged. The electrical conduction is preferably separated by electrically non-conductive coupling elements. With galvanic isolation, electrical potentials are separated from each other and the circuits are then potential-free with respect to each other. This separation must not be stopped at any other point, for example via earthing. The transformer may have a primary winding and a secondary winding. If an AC voltage is now applied to the primary circuit and thus to the primary winding, an alternating current can be formed in the primary winding, which can generate an alternating magnetic field, which in turn can induce an AC voltage in the secondary winding that is then applied to the secondary circuit. In other words, the AC voltage that can be applied to the primary circuit can be transferred from the primary circuit to the secondary circuit by means of the transformer. If necessary, the number of turns of the secondary winding can be greater than the number of turns of the primary winding such that the transformer can step up the AC voltage when transferring the AC voltage from the primary circuit to the secondary circuit in order to obtain a sufficiently high voltage applied to the secondary circuit for a corresponding high-frequency application, in particular for tissue sealing. However, the number of turns of the primary winding can also substantially correspond to the number of turns of the secondary winding. According to the invention, the high-frequency generator further comprises a voltage conversion device which is designed to generate a high-frequency square-wave voltage from the AC voltage transmitted from the primary circuit to the secondary circuit by means of the transformer, which high-frequency square-wave voltage can be tapped at the secondary circuit, in particular at the high-frequency apparatus or electrosurgical instrument or can be applied between electrodes of the electrode pair. In other words, the high-frequency generator is a high-frequency square-wave generator. The voltage conversion device, which can be a converter, generates a high-frequency square-wave voltage from the AC voltage. The AC voltage applicable to the primary circuit can be sinusoidal or rectangular. The AC voltage can have a lower effective value or peak value than the square-wave voltage. The frequency of the AC voltage can also be lower than the frequency of the square-wave voltage. The shape, size and frequency of the AC voltage can be selected in such a way that as few harmonics as possible are generated and, in particular, as few power losses as possible occur in the transformer. It is essential that the voltage conversion device subsequently transforms the AC voltage into a high-frequency square-wave voltage, which can then be tapped at the secondary circuit, in particular applied to the high-frequency apparatus or electrosurgical instrument or between the electrodes and, if necessary, used for coagulation or for tissue cutting. The advantage of using a square-wave signal is that it can be measured more precisely than a sinusoidal signal, which can improve and facilitate the control of the power generated or tapped by the high-frequency generator, in particular the power introduced into the tissue. This makes it possible, in particular, to achieve comparatively higher tissue sealing quality. In addition, carbonization and/or eschar formation can be avoided in this way. As a result, the high-frequency generator according to the invention thus overcomes the disadvantages of the prior art. In particular, the high-frequency generator according to the invention thus enables improved execution of coagulation and/or tissue cutting.

Advantageously, a high-frequency apparatus, in particular an electrosurgical instrument for coagulating and/or cutting biological tissue, can thus be connectable to the secondary circuit such that the high-frequency square-wave voltage can be applicable to the high-frequency apparatus.

In an advantageous embodiment of the invention, the voltage conversion device can comprise a rectifier integrated in the secondary circuit for converting the AC voltage transmitted from the primary circuit to the secondary circuit by means of the transformer into a rectified working voltage and can comprise at least one H-bridge circuit which is integrated in the secondary circuit and to which the working voltage can be applied, wherein the H-bridge circuit can have four electronic switches and a bridge branch to which the high-frequency apparatus or electrosurgical instrument or electrode pair can be connectable, wherein the voltage conversion device can comprise at least one square-wave generator which can be set up to control the switches in such a way that the high-frequency square-wave voltage can be formable in the bridge branch or between the electrodes. Accordingly, the high-frequency square-wave voltage can be tapped at the bridge branch. The H-bridge circuit, which is also called an H-bridge, full bridge or four-quadrant converter, allows for the particularly simple generation of the high-frequency square-wave voltage.

In one embodiment of the invention, the voltage conversion device can comprise a plurality of H-bridge circuits, preferably connected in parallel with one another, in particular two or three H-bridge circuits, wherein the H-bridge circuits can each have four electronic switches and one bridge branch to each of which an electrode pair of the instrument can be connectable, wherein the switches can be controllable by means of the square-wave generator in such a way that the high-frequency square-wave voltage can be formable in each of the bridge branches or between respective electrodes of each electrode pair, preferably independently of one another. The high-frequency square-wave voltage can be tapped at each of the bridge branches. With little effort, a tapped power, especially that introduced into the tissue, can thus be controlled and, if necessary, measured in a plurality of bridge branches or electrode pairs simultaneously and independently of one another. This embodiment can advantageously be used in particular for an electrosurgical instrument as described in the applicant's German patent application with the official file reference 10 2022 125 714.2, which had not yet been published by the priority date of the present patent application. The number of H-bridge circuits can correspond to the number of electrode pairs, so that exactly one H-bridge circuit can be assigned to each electrode pair. Furthermore, it is conceivable that each H-bridge circuit can be controlled by means of its own square-wave generator. The number of square-wave generators can correspond to the number of H-bridge circuits so that exactly one square-wave generator can be assigned to each H-bridge circuit.

Advantageously, the switches can be designed as transistors, in particular bipolar transistors or field-effect transistors (FET), in particular insulated gate field-effect transistors (IGFET), in particular metal oxide semiconductor field effect transistors (MOSFET).

Advantageously, the square-wave generator can be configured to generate pulse width modulated (PWM) signals. By varying the duty cycle and the frequency of the pulse width modulated signals, the frequency of the square-wave voltage and the power introduced into the tissue can then be changed particularly easily.

The working voltage can in principle be adapted to the corresponding high-frequency application. Advantageously, the working voltage can be at least 100 V, preferably at least 200 V, particularly preferably at least 300 V, in order to enable tissue sealing. The working voltage can also be 300 V in particular. The working voltage is a DC voltage.

Advantageously, in addition to the working voltage that can be applied to the H-bridge circuit, a supply voltage can be applicable to the H-bridge circuit and can have a value that is harmless to humans. The supply voltage can be a DC voltage. The supply voltage can be small compared to the working voltage. For example, the supply voltage can be 9 V. To generate the supply voltage, a DC voltage source, which can be part of the secondary circuit, can be connected in parallel with the H-bridge circuit. Preferably, the DC voltage source can be protected from damage caused by the comparatively high working voltage by means of a diode in the secondary circuit.

The frequency of the high-frequency square-wave voltage can in principle be adapted to the corresponding high-frequency application. Advantageously, the high-frequency square-wave voltage can have a frequency of at least 150 kHz, preferably of at least 200 kHz, particularly preferably of at least 300 kHz. This can prevent stimulation of a patient's nerves.

Advantageously, the high-frequency generator can comprise an AC voltage generator for generating the AC voltage. The AC voltage generator, the transformer and the rectifier can form a DC voltage generator.

In an advantageous embodiment of the invention, the AC voltage generator can comprise an H-bridge circuit integrated in the primary circuit, to which a supply voltage can be applicable, wherein the H-bridge circuit can have four electronic switches, wherein the AC voltage generator can comprise a control device which can be set up to control the switches in such a way that it is possible to generate the AC voltage. This design of the AC voltage generator advantageously makes it possible to be able to provide a safety system, which will be described further below, in the high-frequency generator in a particularly simple manner. The switches can be designed as transistors, in particular bipolar transistors or field-effect transistors (FET), in particular insulated gate field-effect transistors (IGFET), in particular metal oxide semiconductor field-effect transistors (MOSFET). Furthermore, the supply voltage, which can advantageously be a DC voltage, can be, for example, 12 V to 24 V, in particular 12 V. The supply voltage can be generated by means of a voltage source, which can be part of the primary circuit. Furthermore, the supply voltage can be small compared to the working voltage. The control device can be a control logic or a driver. Furthermore, the control device can carry out clock processing. A delta modulation method can be used. The control device can be formed, for example, by a processor, in particular a computer. In principle, however, it is also conceivable to apply a mains voltage to the primary circuit, wherein the mains voltage can then optionally be applied to the primary circuit via a transformer.

Advantageously, the high-frequency generator can comprise a measuring device for measuring a voltage formed in the secondary circuit or between the electrodes or the high-frequency square-wave voltage formed in the secondary circuit or between the electrodes and/or a current formed in the secondary circuit or between the electrodes, wherein for this purpose in particular the voltage or square-wave voltage at the bridge branch or the current therein can be detected. The measurement can be performed directly or indirectly. For example, a clamp-on ammeter can be provided as the measuring device. The measuring device can generate a signal, in particular a signal that is proportional to the voltage or current.

In an advantageous embodiment of the invention, it is possible for a signal to be output by means of the measuring device, wherein the high-frequency generator can comprise a processing device which can be set up to receive the signal, to use the signal to conclude an operating state, in particular a correct or incorrect one, of the voltage conversion device or of the H-bridge circuit integrated in the secondary circuit, and to generate a signal describing the operating state.

Advantageously, the signal describing the operating state can then be output by means of the processing device, wherein the AC voltage generator or the control device can be set up to receive the signal describing the operating state and, if the operating state is correct, to initiate the generation of the AC voltage and/or to maintain the generation of the AC voltage and/or, if the operating state is incorrect, not to initiate the generation of the AC voltage and/or to abort the generation of the AC voltage. In the event of a defect in the voltage conversion device or the H-bridge circuit integrated in the secondary circuit, the application of a comparatively high voltage to the secondary circuit can be avoided. The measuring device and the processing device can therefore form a safety system for the high-frequency generator. The signal output by means of the measuring device to the processing device and/or the signal describing the operating state and output by means of the processing device to the AC voltage generator or the control device can be transmitted by means of an optocoupler, which is an electrically non-conductive coupling element.

In one embodiment of the invention, the processing device may comprise an AND gate. This is particularly advantageous if the voltage conversion device comprises a plurality of H-bridge circuits, wherein, for example, the voltage or the current can then be measured at or in each bridge branch by means of the measuring device, wherein corresponding signals generated by means of the measuring device can be transmitted to the processing device or the AND gate. If at least one H-bridge circuit is in an incorrect operating state, for example in the event of a defect, this can be detected by the processing device or the AND gate, wherein the signal from the processing device then describing the incorrect operating state can cause the AC voltage generator or the control device not to initiate the generation of the AC voltage or to abort the generation of the AC voltage.

Further advantageous embodiments of the high-frequency generator can be found in the feature descriptions of the dependent claims referring back to method claim 17.

The system according to the invention comprises a high-frequency generator according to the invention and a high-frequency apparatus, in particular an electrosurgical instrument for coagulating and/or cutting biological tissue, which is connected to a secondary circuit of the high-frequency generator.

The instrument may comprise at least one pair of electrodes which may be connected to the secondary circuit such that the secondary circuit may be closable via the tissue.

Furthermore, the instrument can be monopolar or bipolar. The electrode pair can then comprise at least one active electrode and one neutral electrode or at least two active electrodes. Advantageously, however, the instrument is bipolar.

In the method according to the invention for generating a high-frequency square-wave voltage by means of a high-frequency generator, an AC voltage is applied to a primary circuit of the high-frequency generator, wherein a secondary circuit of the high-frequency generator is coupled to the primary circuit in a manner galvanically isolated from the primary circuit by means of a transformer of the high-frequency generator, wherein the high-frequency square-wave voltage is generated by means of a voltage conversion device of the high-frequency generator from the AC voltage transmitted from the primary circuit to the secondary circuit by means of the transformer.

For the advantageous effects of the method according to the invention, reference is made to the description of the advantages of the high-frequency generator according to the invention.

A high-frequency apparatus or electrosurgical instrument or at least one pair of electrodes of the electrosurgical instrument can be connected to the secondary circuit, wherein the high-frequency square-wave voltage can be generated between electrodes of the electrode pair. The high-frequency square-wave voltage can be applied to the high-frequency apparatus or electrosurgical instrument.

In an advantageous embodiment of the method, by means of a rectifier of the voltage conversion device integrated in the secondary circuit, the AC voltage transmitted from the primary circuit to the secondary circuit by means of the transformer can be converted into a rectified working voltage, wherein the working voltage can be applied to at least one H-bridge circuit integrated in the secondary circuit, wherein the high-frequency apparatus or the electrosurgical instrument or the electrode pair can be connected to a bridge branch of the H-bridge circuit, wherein four electronic switches of the H-bridge circuit can be controlled by means of at least one square-wave generator of the voltage conversion device in such a way that the high-frequency square-wave voltage can be formed in the bridge branch or between the electrodes.

Advantageously, when the high-frequency generator or high-frequency apparatus or electrosurgical instrument is started up, a supply voltage can first be applied to the H-bridge circuit integrated in the secondary circuit and can have a value that is safe for humans, wherein a voltage formed in the bridge branch or between the electrodes and/or a current formed in the bridge branch or between the electrodes can be measured by means of a measuring device of the high-frequency generator, wherein a signal can be output by means of the measuring device, wherein the signal can be received by means of a processing device of the high-frequency generator, a correct or incorrect operating state of the H-bridge circuit integrated in the secondary circuit can be concluded on the basis of the signal and a signal describing the operating state can be generated, wherein the signal describing the operating state can be output by means of the processing device, wherein the signal describing the operating state can be received by means of a control device of an AC voltage generator of the high-frequency generator and four electronic switches of an H-bridge circuit of the AC voltage generator integrated in the primary circuit can be controlled on the basis of the signal describing the operating state such that if the operating state is correct, the generation of the AC voltage can be initiated and/or if the operating state is incorrect, the generation of the AC voltage cannot be initiated.

Advantageously, after starting up, when operating the high-frequency generator or high-frequency apparatus or electrosurgical instrument, the working voltage can be applied to the H-bridge circuit integrated in the secondary circuit in addition to the supply voltage, wherein the four electronic switches of the H-bridge circuit integrated in the primary circuit can be controlled by means of the control device on the basis of the signal describing the operating state such that if the operating state is correct, the generation of the AC voltage can be maintained and/or if the operating state is incorrect, the generation of the AC voltage can be aborted so that after the generation of the AC voltage has been aborted, only the supply voltage can continue to be applied to the H-bridge circuit integrated in the secondary circuit. In the event of a defect, the voltage applied to the secondary circuit can thus drop to the level that is safe for humans.

Further advantageous embodiments of the method can be found in the feature descriptions of the dependent claims referring back to device claim 1.

According to the invention, a high-frequency generator according to the invention is used for an electrosurgical instrument for coagulating and/or cutting a biological tissue.

For the advantageous effects of the use according to the invention, reference is made to the description of the advantages of the high-frequency generator according to the invention.

Further advantageous embodiments of the use can be found in the feature descriptions of the dependent claims referring back to device claim 1 and method claim 17.

### BRIEF DESCRIPTION OF THE DRAWINGS

Preferred embodiments of the invention will be explained in more detail below with reference to the accompanying drawings.

Shown are:

FIG. 1 a circuit diagram of a high-frequency generator in one embodiment;

FIG. 2 a block diagram of a high-frequency generator in a further embodiment.

### DETAILED DESCRIPTION

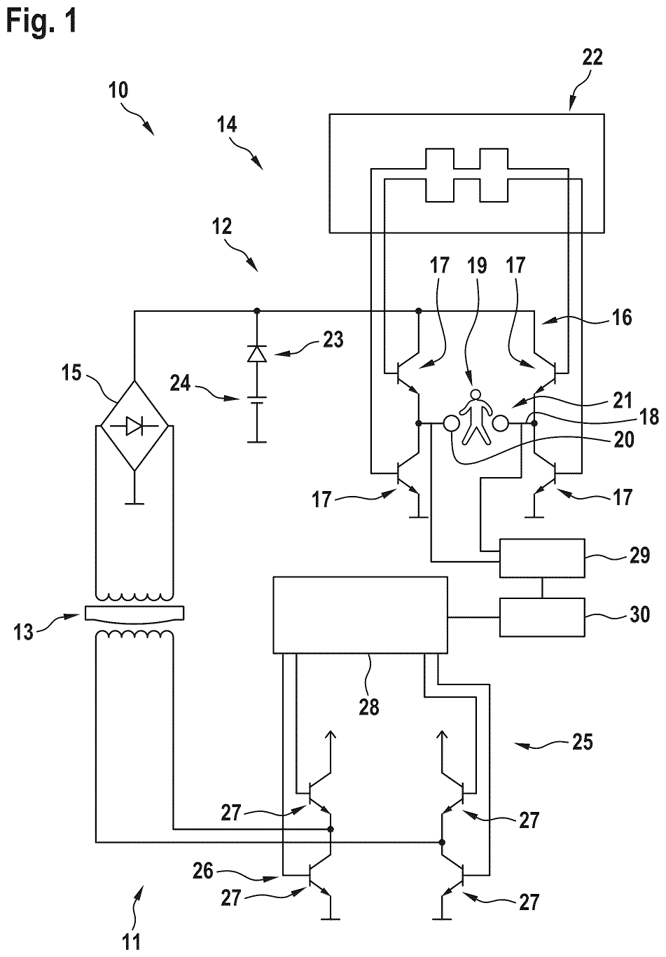

FIG. 1 shows a circuit diagram of a high-frequency generator 10 for an electrosurgical instrument (not shown in detail here) for coagulating or cutting biological tissue, comprising a primary circuit 11 to which an AC voltage can be applied, a secondary circuit 12, a transformer 13 for coupling the primary circuit 11 to the secondary circuit 12 in a manner galvanically isolated from the secondary circuit 12, and a voltage conversion device 14 which is designed to generate a high-frequency square-wave voltage having a frequency of at least 150 kHz from the AC voltage transmitted from the primary circuit 11 to the secondary circuit 12 by means of the transformer 13.

The voltage conversion device 14 comprises a rectifier 15 integrated in the secondary circuit 12 for converting the AC voltage transmitted by means of the transformer 13 from the primary circuit 11 to the secondary circuit 12 into a rectified working voltage and an H-bridge circuit 16 which is integrated in the secondary circuit 12 and to which the working voltage can be applied, wherein the H-bridge circuit 16 has four electronic switches 17 designed as transistors and a bridge branch 18 to which electrodes 20 of an electrode pair 21 of the instrument that are provided for the treatment of a patient 19 are connected, wherein the voltage conversion device 14 comprises a square-wave generator 22 which is set up to control the switches 17 in such a way that the high-frequency square-wave voltage can be formed between the electrodes 20. The square-wave generator 22 is designed to generate pulse width modulated (PWM) signals, which control the switches 17.

In the present example, the working voltage is 300 V. In addition to the working voltage that can be applied to the H-bridge circuit 16, a supply voltage can be applied to the H-bridge circuit 16 via a voltage source 24 protected from the working voltage by means of a diode 23, which supply voltage has a value that is harmless to humans, which in the present example is 9 V.

Furthermore, the high-frequency generator 10 comprises an AC voltage generator 25 for generating the AC voltage, which comprises an H-bridge circuit 26 integrated in the primary circuit 11, to which a supply voltage can be applied, which in the present example is 12 V, wherein the H-bridge circuit 26 has four electronic switches 27 designed as transistors, wherein the AC voltage generator 25 comprises a control device 28 which is set up to control the switches 27 in such a way that the AC voltage can be generated.

Furthermore, the high-frequency generator 10 comprises a measuring device 29 for measuring a voltage formed between the electrodes 20 or a current formed between the electrodes 20. A signal can be output by means of the measuring device 29, wherein the high-frequency generator 10 comprises a processing device 30 which is set up to receive the signal, to use the signal to conclude an operating state, in particular a correct or incorrect operating state, of the voltage conversion device 14, in particular a defect in the H-bridge circuit 16, and to generate a signal describing the operating state. The signal describing the operating state can be output by means of the processing device 30, wherein the AC voltage generator 25 or the control device 28 is set up to receive the signal describing the operating state and, if the operating state is correct, to initiate the generation of the AC voltage and/or to maintain the generation of the AC voltage, or, if the operating state is incorrect, not to initiate the generation of the AC voltage and/or to abort the generation of the AC voltage. In particular, the connection of the measuring device 29 to the processing device 30 and the connection of the processing device 30 to the control device 28 is shown here only schematically.

The high-frequency generator 10 has been described above in connection with an electrosurgical instrument. However, the high-frequency generator 10 can in principle also be used in connection with other high-frequency applications. In particular, another high-frequency apparatus can be connected to the bridge branch 18.

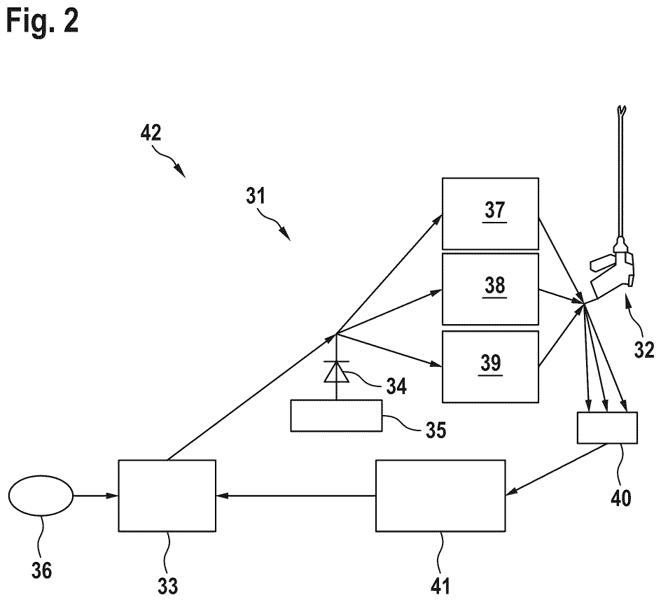

FIG. 2 shows a block diagram of a high-frequency generator 31 for an electrosurgical instrument 32 for coagulating or cutting biological tissue. A working voltage generated by a DC voltage generator 33 of the high-frequency generator 31, wherein the DC voltage generator 33 can be formed in particular by an AC voltage generator, a transformer and a rectifier, which working voltage in the present example is 400 V, can be applied together with a supply voltage supplied by a voltage source 35 protected from the working voltage by a diode 34, which supply voltage in the present example is 9 V, to a voltage conversion device (not shown in detail here) of the high-frequency generator 31. The DC voltage generator 33 is supplied with power by a voltage source 36, which in the present example provides a supply voltage of 12 V to 24 V. The voltage conversion device comprises three H-bridge circuits (not shown here), wherein each H-bridge circuit can be controlled by means of its own square-wave generator 37, 38, 39 of the voltage conversion device, which are each set up to generate pulse width modulated (PWM) signals with a frequency of 300 kHz, in order to generate a high-frequency square-wave voltage from a voltage that can be applied to the corresponding H-bridge circuit, independently of the other two H-bridge circuits in each case. Each of the three independently controllable high-frequency square-wave voltages can be applied to a corresponding electrode pair of three electrode pairs (not shown here) of the instrument 32. In particular, by means of an AND gate 40 of a processing device (not shown in detail here) of the high-frequency generator 31, a correct or incorrect operating state of the voltage conversion device or the H-bridge circuits is determined on the basis of three signals generated by a measuring device (not shown here) of the high-frequency generator 31, which signals result from the measurement of a voltage or a current at or in each bridge branch of the H-bridge circuits, wherein the processing device generates a signal describing the operating state and transmits this to a control device 41 of the DC voltage generator 33, which is illustrated in FIG. 2 by a separate block separated from a block illustrating the DC voltage generator 33, which initiates, maintains or aborts the generation of the working voltage according to the signal describing the operating state.

The high-frequency generator 31 and the instrument 32 are part of a system 42.

The high-frequency generator 31 has been described above in connection with an electrosurgical instrument or the electrosurgical instrument 32. However, the high-frequency generator 31 can in principle also be used in connection with other high-frequency applications or other high-frequency apparatuses.

The invention relates to a high-frequency generator 10) comprising a primary circuit 11) to which an AC voltage can be applied, a secondary circuit 12), a transformer 13) for coupling the primary circuit to the secondary circuit in a manner galvanically isolated from the secondary circuit, and a voltage conversion device 14) which is designed to generate a high-frequency square-wave voltage from the AC voltage transmitted from the primary circuit to the secondary circuit by means of the transformer.

### LIST OF REFERENCE SIGNS

10 High-frequency generator
11 Primary circuit
12 Secondary circuit
13 Transformer
14 Voltage conversion device
15 Rectifier
16 H-bridge circuit
17 Electronic switch
18 Bridge branch
19 Patient
20 Electrode
21 Electrode pair
22 Square-wave generator
23 Diode
24 Voltage source
25 AC voltage generator
26 H-bridge circuit
27 Electronic switch
28 Control device
29 Measuring device
30 Processing device
31 High-frequency generator
32 Electrosurgical instrument
33 DC voltage generator
34 Diode
35 Voltage source
36 Voltage source
37 Square-wave generator
38 Square-wave generator
39 Square-wave generator
40 AND gate
41 Control device
42 System

1. A high-frequency generator, comprising a primary circuit to which an AC voltage can be applied, a secondary circuit, a transformer for coupling the primary circuit to the secondary circuit in a manner galvanically isolated from the secondary circuit, and a voltage conversion device which is designed to generate a high-frequency square-wave voltage from the AC voltage transmitted from the primary circuit to the secondary circuit by means of the transformer.

2. The high-frequency generator according to claim 1, wherein
a high-frequency apparatus, in particular an electrosurgical instrument for coagulating and/or cutting biological tissue, can be connected to the secondary circuit such that the high-frequency square-wave voltage can be applied to the high-frequency apparatus.

3. The high-frequency generator according to claim 1, wherein
the voltage conversion device comprises a rectifier integrated in the secondary circuit for converting the AC voltage transmitted from the primary circuit to the secondary circuit by means of the transformer into a rectified working voltage and comprises at least one H-bridge circuit which is integrated in the secondary circuit and to which the working voltage can be applied, the H-bridge circuit having four electronic switches and a bridge branch, the voltage conversion device comprising at least one square-wave generator (22, 37, 38, 39) which is set up to control the switches in such a way that the high-frequency square-wave voltage can be formed in the bridge branch.

4. The high-frequency generator according to claim 3, wherein
the voltage conversion device comprises a plurality of H-bridge circuits, preferably connected in parallel with one another, in particular two or three H-bridge circuits, the H-bridge circuits each having four electronic switches and one bridge branch, the switches being controllable by means of the square-wave generator in such a way that the high-frequency square-wave voltage can be formed in each of the bridge branches, preferably independently of one another.

5. The high-frequency generator according to claim 3, wherein
the switches are designed as transistors, in particular bipolar transistors or field-effect transistors (FET), in particular insulated gate field-effect transistors (IGFET), in particular metal oxide semiconductor field-effect transistors (MOSFET).

6. The high-frequency generator according to claim 3, wherein
the square-wave generator is designed to generate pulse width modulated (PWM) signals.

7. The high-frequency generator according to claim 3, wherein
the working voltage is at least 100 V, preferably at least 200 V, particularly preferably at least 300 V.

8. The high-frequency generator according to claim 3, wherein
a supply voltage can be applied to the H-bridge circuit in addition to the working voltage that can be applied to the H-bridge circuit.

9. The high-frequency generator according to claim 1, wherein
the high-frequency square-wave voltage has a frequency of at least 150 kHz, preferably of at least 200 kHz, particularly preferably of at least 300 kHz.

10. The high-frequency generator according to claim 1, wherein
the high-frequency generator comprises an AC voltage generator for generating the AC voltage.

11. The high-frequency generator according to claim 10, wherein
the AC voltage generator comprises an H-bridge circuit integrated in the primary circuit, to which a supply voltage can be applied, the H-bridge circuit having four electronic switches, the AC voltage generator comprising a control device which is designed to control the switches in such a way that the AC voltage can be generated.

12. The high-frequency generator according to claim 1, wherein
the high-frequency generator comprises a measuring device for measuring a voltage formed in the secondary circuit and/or a current formed in the secondary circuit.

13. The high-frequency generator at least according to claim 10, wherein
a signal can be output by means of the measuring device, the high-frequency generator comprising a processing device which is designed to receive the signal, to use the signal to conclude an operating state, in particular a correct or incorrect operating state, of the voltage conversion device, and to generate a signal describing the operating state.

14. The high-frequency generator according to claim 13, wherein
the signal describing the operating state can be output by means of the processing device, the AC voltage generator being set up to receive the signal describing the operating state and, if the operating state is correct, to initiate the generation of the AC voltage and/or to maintain the generation of the AC voltage and/or, if the operating state is incorrect, not to initiate the generation of the AC voltage and/or to abort the generation of the AC voltage.

15. The high-frequency generator according to claim 13, wherein
the processing device has an AND gate.

16. A system comprising a high-frequency generator according to claim 1, and a high-frequency apparatus, in particular an electrosurgical instrument for coagulating and/or cutting biological tissue, which is connected to a secondary circuit of the high-frequency generator.

17. A method for generating a high-frequency square-wave voltage by means of a high-frequency generator, wherein an AC voltage is applied to a primary circuit of the high-frequency generator, wherein a secondary circuit of the high-frequency generator is coupled to the primary circuit by means of a transformer (13) of the high-frequency generator in a manner galvanically isolated from the primary circuit, wherein the high-frequency square-wave voltage is generated by means of a voltage conversion device (14) of the high-frequency generator from the AC voltage transmitted from the primary circuit to the secondary circuit by means of the transformer.

18. The method according to claim 17, wherein
by means of a rectifier of the voltage conversion device integrated in the secondary circuit, the AC voltage transmitted from the primary circuit to the secondary circuit by means of the transformer is transformed into a rectified working voltage, the working voltage being applied to at least one H-bridge circuit integrated in the secondary circuit, four electronic switches of the H-bridge circuit being controlled by means of at least one square-wave generator of the voltage conversion device such that the high-frequency square-wave voltage is formed in a bridge branch of the H-bridge circuit.

19. The method according to claim 18, wherein
when the high-frequency generator is started up, a supply voltage is first applied to the H-bridge circuit integrated in the secondary circuit, a voltage formed in the bridge branch and/or a current formed in the bridge branch being measured by means of a measuring device of the high-frequency generator, a signal being output by means of the measuring device, the signal being received by means of a processing device of the high-frequency generator, a correct or incorrect operating state of the H-bridge circuit integrated in the secondary circuit being concluded on the basis of the signal, and a signal describing the operating state being generated, the signal describing the operating state being output by means of the processing device, the signal describing the operating state being received by means of a control device of an AC voltage generator of the high-frequency generator, and four electronic switches of an H-bridge circuit of the AC voltage generator integrated in the primary circuit being controlled on the basis of the signal describing the operating state such that if the operating state is correct, the generation of the AC voltage is initiated and/or if the operating state is incorrect, the generation of the AC voltage is not initiated.

20. The method according to claim 19, wherein
after starting up, during operation of the high-frequency generator, the working voltage is applied to the H-bridge circuit integrated in the secondary circuit in addition to the supply voltage, the four electronic switches of the H-bridge circuit integrated in the primary circuit being controlled by means of the control device on the basis of the signal describing the operating state such that if the operating state is correct, the generation of the AC voltage is maintained and/or if the operating state is incorrect, the generation of the AC voltage is aborted, and therefore after the generation of the AC voltage is aborted, only the supply voltage continues to be applied to the H-bridge circuit integrated in the secondary circuit.

21. (canceled)
